(19) United States
(12) Patent Application Publication
(10) Pub. No.: US 2016/0287912 A1
(43) Pub. Date: Oct. 6, 2016

(54) METHOD AND APPARATUS FOR PERFORMANCE OF THERMAL BRONCHIPLASTY WITH UNFOCUSED ULTRASOUND

(71) Applicant: GUIDED INTERVENTIONS, INC., Elyria, OH (US)

(72) Inventor: Reinhard J. WARNKING, Setauket, NY (US)

(21) Appl. No.: 15/038,652

(22) PCT Filed: Oct. 31, 2014
(86) PCT No.: PCT/US2014/063344

§ 371 Date: May 23, 2016

Related U.S. Application Data
(60) Provisional application No. 61/899,568, filed on Nov. 4, 2013.
(60) Provisional application No. 61/899,958, filed on Nov. 5, 2013.

(51) Int. Cl. A61N 7/02; A61B 8/08; A61B 1/267; A61B 8/00; A61B 8/12; A61B 8/14

(52) U.S. Cl. CPC A61N 7/022; A61B 8/12; A61B 8/14; A61B 1/2676; A61B 8/4483; A61B 8/08; A61N 2007/0052

(57) ABSTRACT

Apparatus and methods for deactivating bronchial nerves and smooth muscle extending along a bronchial branch of a mammalian subject to treat asthma and related conditions. An electromechanical transducer (11) is inserted into the bronchus as, for example, by advancing the distal end of a catheter (10) bearing the transducer into the bronchial section to be treated. The electromechanical transducer emits unfocused mechanical vibratory energy of one or more ultrasonic frequencies so as to heat tissues throughout a relatively large target region (13) as, for example, at least about 1 cm³ encompassing the bronchus to a temperature sufficient to inactivate nerves but insufficient to cause rapid ablation or necrosis of organic tissues. The treatment can be performed without locating or focusing on individual bronchial nerves.

### CROSS-REFERENCE TO RELATED APPLICATION

This application claims the benefit of U.S. Provisional Patent Applications No. 61/899,958 filed Nov. 5, 2013, and 61/899,568 filed Nov. 4, 2013.

### BACKGROUND OF THE INVENTION

Successful treatment of pulmonary diseases such as asthma or COPD is important since these diseases represent a significant global health issue with reduced quality of life. While drug therapy (Bronchodilators, Anti-inflammatories and Leukotriene Modifiers) can be used to treat asthma, it is not always successful and very expensive. Asthma and COPD are disorders that are characterized by airway constriction and inflammation resulting in breathing difficulties. Wheezing, shortness of breath and coughing are typical symptoms.

These symptoms are caused by increased mucus production, airway inflammation and smooth muscle contraction, resulting in airway obstruction. This obstruction can be treated by injuring and scaring the bronchial walls. This remodeling of the bronchial walls stiffens the bronchia and reduces contractility. Mechanical means and heat application have been proposed as in U.S. Pat. No. 8,267,094 B2. Other approaches focus on destruction of smooth muscle cells surrounding the bronchia as described in US 2012/0143099A1 and U.S. Pat. No. 7,906,124B2. Others describe applying RF energy to the bronchial wall and thereby directly widening the bronchia through a process which is not disclosed as in U.S. Pat. No. 7,740,017B2 and U.S. Pat. No. 8,161,978B2. Whatever the process, the bronchial wall will be damaged and the procedure therefore has to be staged as described in U.S. Pat. No. 7,740,017B2. EP2405841 describes applications of heat shocks through infused agents.

Inactivating conduction of the nerves surrounding the bronchia has been proposed, in US Patent Application Publication No. 2012/0203216A1, through mechanical action, i.e., puncturing, tearing, cutting nerve tissue. In US 2011/0118725 nerve tissue ablation is proposed by applying energy (RF, HIFU, Microwave, Radiation and Thermal Energy) directly to the nerves percutaneously. It is not taught how to identify the nerve location in order to align the energy focal zone (i.e. HIFU) with the nerve location. This is an issue since nerves are too small to be visualized in vivo with standard ultrasound, CT or MRI imaging methods. Therefore, the focal zone of the energy field cannot be predictably aligned with the target or nerve location. U.S. Pat. No. 8,088,127B2 teaches to denervate by applying RF energy to the bronchial wall with the catheter positioned inside the bronchial lumen. It is proposed to protect the bronchial wall through simultaneous cooling of the wall. This is of course a very time intensive treatment approach since the RF ablation is limited to the electrode contact areas. Therefore numerous ablation zones need to be pieced together to obtain a larger ablation zone with increased probability of affecting nerves. Efficacy might be severely limited due to the relatively small treatment areas and maybe the cooling action.

However, how to selectively target predominantly nerves or smooth muscle without affecting bronchial wall and surrounding tissue is not being taught. There is a need for a device and method to selectively ablate bronchial nerves without causing damage to bronchial walls and surrounding tissues. If this can be achieved, treatments would be much easier and faster to perform. Today's multiple treatments (see U.S. Pat. No. 7,740,017B2 and Alair System description, BSX) can be reduced to a one time treatment much better tolerated by the patient. By selectively targeting nerves instead of tissue it is also likely that a more proximal single ablation of nerves (conducting signals to distal bronchial sections) will have the same clinical effect as treating the bronchial tree from proximal to distal with numerous energy applications.

In order to explain the difficulties associated with accomplishing this task without causing other damage, the anatomy of the bronchial system and nerves will be described now. Shown in FIG. 6 is an illustration of the bronchial tree (1). FIG. 3 shows a cross section of a bronchial tube surrounded with smooth muscle (7) and nerves (6). In addition, FIG. 5 shows a longitudinal section of a bronchus (1) and the adjacent nerves (6). As can be seen from these two FIGS. 3 and 5), the bronchial nerves (6) surround the bronchial tubes. Different individuals have the nerves (6) in different locations around the bronchial tubes. Thus, the nerves may be at different radial distances from the central axis where the energy emitter (11) is placed (FIG. 3). The nerves also may be at different locations around the circumference of the bronchial tubes. It is not practical to locate the bronchial nerves by referring to anatomical landmarks. Moreover, it is difficult or impossible to locate individual bronchial nerves using common in vivo imaging technology.

The inability to locate and target the bronchial nerves (6) makes it difficult to disconnect the bronchial nerve activity using non-surgical techniques without causing damage to the bronchial walls or causing other side effects. For example, attempts to apply energy to the bronchial nerves can cause effects such as stenosis, and necrosis. In addition, the inability to target and locate the bronchial nerves (6) makes it difficult to ensure that bronchial nerve activity has been discontinued enough to achieve an acceptable therapeutic treatment.

U.S. Pat. No. 8,088,127B2 suggests the use of a radio frequency (“RF”) emitter connected to a catheter, which is inserted in the bronchial tree. The RF emitter is placed against the bronchial wall and the RF energy is emitted to heat the nerves to a temperature that reduces the activity of bronchial nerves which happen to lie in the immediate vicinity of the emitter. In order to treat all the nerves surrounding the bronchial tubes, the RF emitter source must be repositioned around the inside of each bronchial tube section multiple times. In order to protect the bronchial wall this RF heat application is combined with a cooling application which makes the procedure even more complicated. The emitter may miss some of the bronchial nerves, leading to an incomplete treatment. Moreover, the RF energy source (electrode) must contact the bronchial wall to be able to heat the surrounding tissue and nerves, which may cause damage or necrosis to the inner lining of the bronchi despite the proposed cooling mechanism.

The US2011/0118725 Patent application also suggests the use of high-intensity focused ultrasound to deactivate the bronchial nerves. It is not clear how a High Intensity Focused Ultrasound zone can be aligned with the targeted bronchial nerves. It is difficult or impossible to align this highly focused zone with the bronchial nerves because it is difficult or impossible to visualize and target the bronchial nerves with current in vivo imaging technology, and because the bronchial nerves may lie at different radial distances and circumferential locations from the central axis of bronchi. The latter is a problem particularly in patients who have bronchi with large variations in shape or thickness. Moreover, the thin focal zone can encompass only a small segment of each bronchial nerve along the lengthwise direction of the bronchi. Since nerves damage is reversible, a small treatment zone allows the nerves to reconnect in a shorter period of time.

Ultrasound has been used to enhance cell repair, stimulate the growth of bone cells, enhance delivery of drugs to specific tissues, and to image tissue within the body. Recently, high-intensity focused ultrasound has been used to heat and ablate tumors and tissue within the body. Ablation of tissue has been performed nearly exclusively by high-intensity focused ultrasound because the emitted ultrasonic mechanical vibratory energy is focused on a specific location to allow precise in-depth tissue necrosis without affecting surrounding tissue and intervening structures that the ultrasonic mechanical vibratory energy must pass through.

U.S. Pat. No. 6,117,101, to Diederich, discusses use of highly collimated ultrasonic mechanical vibratory energy rather than high intensity focused ultrasound for ablating tissue to create a scar ring within the pulmonary vein for blocking the conduction of electrical signals to the heart.

### BRIEF SUMMARY OF THE INVENTION

One aspect of the invention provides an apparatus for inactivating bronchial nerves in a human or non-human mammalian subject. The apparatus according to this aspect of the invention preferably includes an electromechanical transducer adapted for insertion into the bronchial system of the mammalian subject. The electromechanical transducer desirably is arranged to transmit unfocused ultrasonic mechanical vibratory energy. The apparatus according to this aspect of the invention desirably also includes a generator circuit electrically connected to the transducer. The generator circuit most preferably is adapted to control the electromechanical transducer to transmit unfocused ultrasonic mechanical vibratory energy into an target region of at least approximately 1 cm³, encompassing the bronchial tube so that the unfocused ultrasonic mechanical vibratory energy is applied at a desired therapeutic level sufficient to inactivate conduction of bronchial nerves throughout the target region. As discussed further below, such therapeutic level is well below the level required for tissue ablation.

The apparatus may further include a catheter with a distal end and a proximal end, the transducer being mounted to the catheter adjacent the distal end, the transducer being constructed and arranged inside an inflatable bladder or balloon which will make contact with the bronchial wall. This bladder is filled with a circulating cooling fluid which serves in part to conduct ultrasonic mechanical vibratory energy from the transducer to the bronchial walls and surrounding tissue and nerves. This cooling fluid also transports excessive heat away from the transducer. About half of the electrical energy supplied to the transducer is converted into heat while roughly the other half is converted to ultrasonic energy. The catheter may have an additional expansible element such as a compliant balloon or a similar anchoring device like an expandable wire basket mounted adjacent the distal end for cooperating with the inflatable transducer-containing bladder to hold the catheter so that a longitudinal axis of the transducer remains generally parallel to the axis of the target bronchial tube section. The transducer may be adapted to transmit the ultrasonic mechanical vibratory energy in a 360° cylindrical pattern surrounding the transducer axis, and the catheter may be constructed and arranged (for instance, with the secondary expansible element) to hold the axis of the transducer generally parallel to the axis of the bronchial tube.

A method according to a further aspect of the invention desirably includes the steps of inserting an electromechanical transducer into a bronchial branch of the subject and energizing the transducer to transmit therapeutically effective unfocused ultrasonic mechanical vibratory energy into an target region of at least approximately 1 cubic centimeter encompassing the bronchial branch. The ultrasonic mechanical vibratory energy is applied with such an amplitude, frequency and duration that the energy inactivates all nerves in the target region. For example, the step of energizing the transducer may be so as to maintain the temperature of the bronchial wall below 65° C. while heating the solid tissues within the target region, including the nerves in the target region, to above 42° C.

Because the target region is relatively large, and because the tissues throughout the target region preferably reach temperatures for a certain time span sufficient to inactivate nerve conduction, the preferred methods according to this aspect of the invention can be performed successfully without determining the actual locations of the bronchial nerves, and without targeting or focusing on the bronchial nerves. The treatment can be performed without measuring the temperature of tissues. Moreover, the treatment preferably is performed without causing injury to the bronchi. The preferred methods and apparatus can inactivate relatively long segments of the bronchial nerves, so as to reduce the possibility of nerve recovery which would re-establish conduction along the inactivated segments.

Further aspects of the invention provide probes which can be used in the method and apparatus discussed above, and apparatus incorporating means for performing the steps of the methods discussed above.

### BRIEF DESCRIPTION OF THE DRAWINGS

FIG. 1 is an anatomical view of typical main bronchial trunks 1 and 2 and associated structures.

FIG. 2 is showing a treatment catheter 10 advanced through a bronchoscope 5 into the right bronchial branch and the diagrammatic sectional view depicting the unfocused ultrasound treatment volume 13.

FIG. 3 shows a cross section through a bronchial tube with an electromechanical transducer 11 in the center surrounded by the cooling fluid in the compliant balloon.

FIG. 4 demonstrates the effects on power distribution of proper alignment vs. a non-centered, non-aligned electromechanical transducer.

FIG. 5 shows a right bronchial branch with adjacent nerves running alongside the bronchial tube.

FIG. 6 shows a bronchial tree in its entirety.

FIG. 7 is a flow chart depicting the steps used in treating the bronchi.

FIG. 8 is a schematic view of a distal end portion of an elongate flexible isometric (constant outer diameter) sheath, showing the placement of a circular ultrasound imaging array at the distal section of the sheath.

FIG. 9A is a schematic view of the distal end portion of the isometric sheath of FIG. 8 inside a heart, showing the sheath as used in a typical medical procedure monitoring a trans-septal puncture.

FIG. 9B is a schematic elevational view of a video monitor or display showing an image of a cardiac septum during the ultrasound-guided procedure of FIG. 9A. Left and right atrium are mixed up.

FIG. 10A is a schematic isometric view of a distal end portion of another sheath monitoring a trans-septal puncture in a heart, the sheath having a longitudinal ultrasound imaging array.

FIG. 10B is a schematic elevational view of a video monitor or display showing an image of a cardiac septum during the ultrasound-guided procedure of FIG. 10A.

FIG. 11 is a view of the imaging sheath of FIG. 8 in a related operating procedure, placed inside the left atrium of a heart and monitoring catheter-mediated ablation at the left superior pulmonary vein (LSPV).

FIG. 12 is a schematic view of a distal end portion of a modified elongate flexible medical sheath, depicting additional ultrasound imaging components mounted into a wall of the isometric sheath.

FIG. 13 is a schematic longitudinal cross-sectional view of a distal end portion of another embodiment of an elongate flexible medical sheath, in accordance with the present invention, showing an annular ultrasound imaging array divided into imaging and therapeutic sections.

FIG. 14 is a schematic perspective view of an imaging/treatment catheter in accordance with the present invention, which is introduced into a patient over a circular (loop) guide wire mapping catheter.

FIG. 15 is a schematic perspective view of the imaging/treatment catheter of FIG. 14 inserted through a sheath and positioned at the left superior pulmonary vein (LSPV) inside the left atrium with a sensing loop at the distal end advanced into the LSPV.

FIG. 16 is a flow chart depicting major steps of a PV isolation process utilizing the instrument of FIGS. 14 and 15.

FIG. 17 is partially a schematic perspective view of the imaging/treatment catheter of FIGS. 14 and 15 and partially a block diagram of a control system connected to the imaging/treatment catheter.

FIG. 18 is a block diagram of selected components of an electronic control unit and image generating components of a computer unit of an apparatus in accordance with the present invention for generating ablation zones of predetermined shape on inner surfaces of hollow internal organs of a mammalian subject.

### DETAILED DESCRIPTION

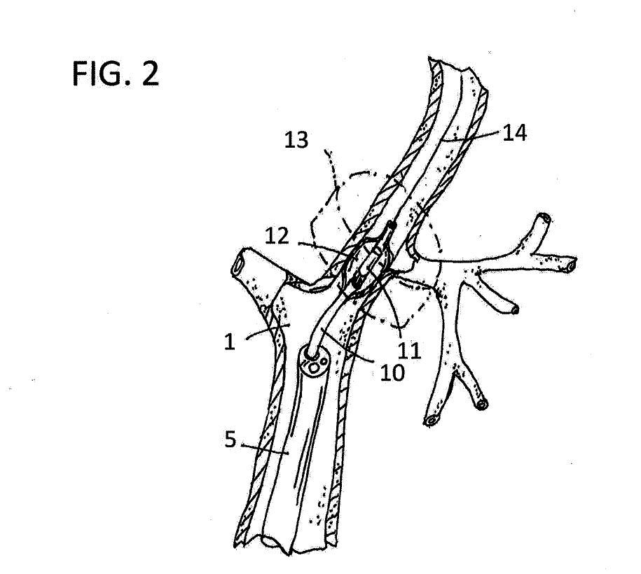

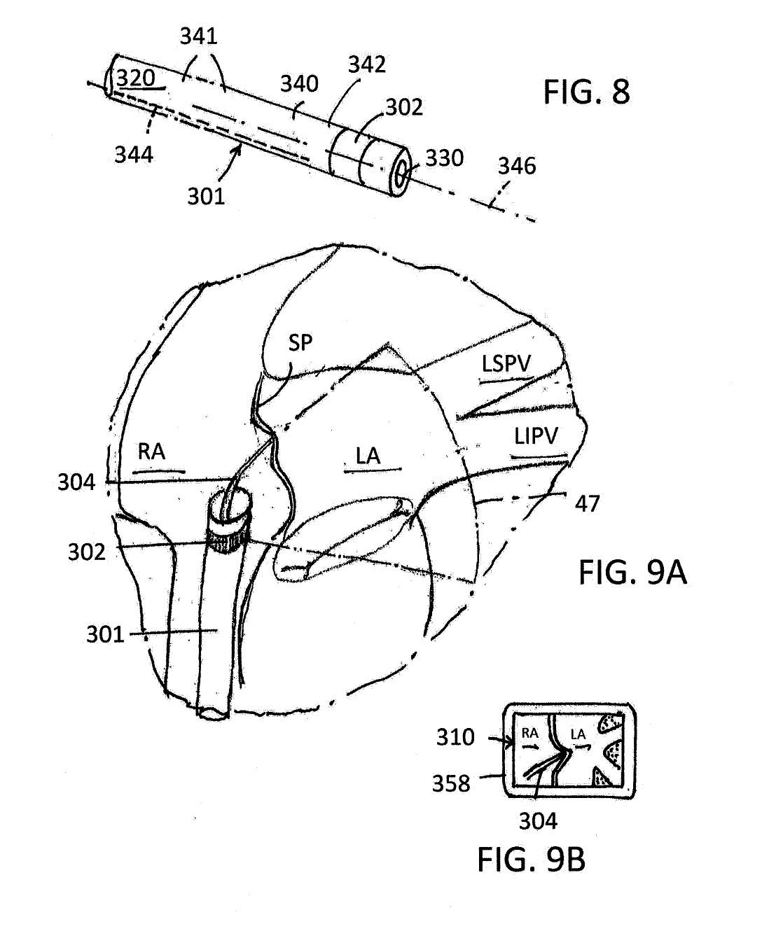

Apparatus according to one embodiment of the invention (FIG. 2) is advanced through the working channel of a bronchoscope 5. Alternatively the catheter can be advanced through a sheath. The sheath generally may be in the form of an elongated tube having a proximal end, a distal end and a proximal-to-distal axis. The sheath may be a steerable sheath. Thus, the sheath may include known elements such as one or more pull wires (not shown) extending between the proximal and distal ends of the sheath and connected to a steering control arranged so that actuation of the steering control by the operator flexes the distal end of the sheath in a direction transverse to the axis. The sheath might be equipped with a circular ultrasound imaging array at the distal portion to allow for image guidance for the denervation procedure (as described in detail hereinafter with reference to FIGS. 8-13.

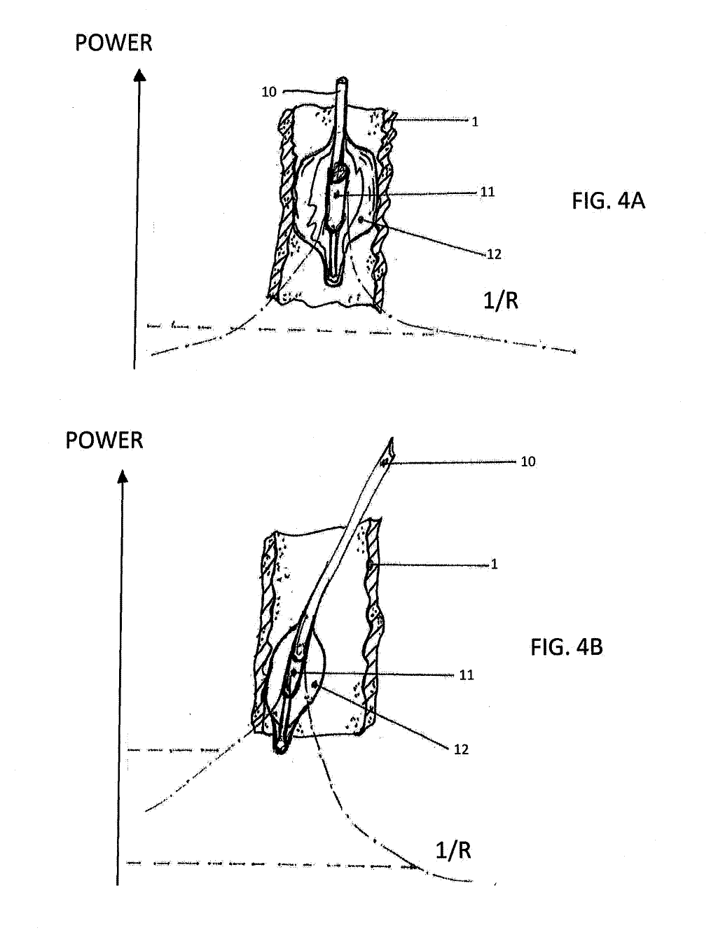

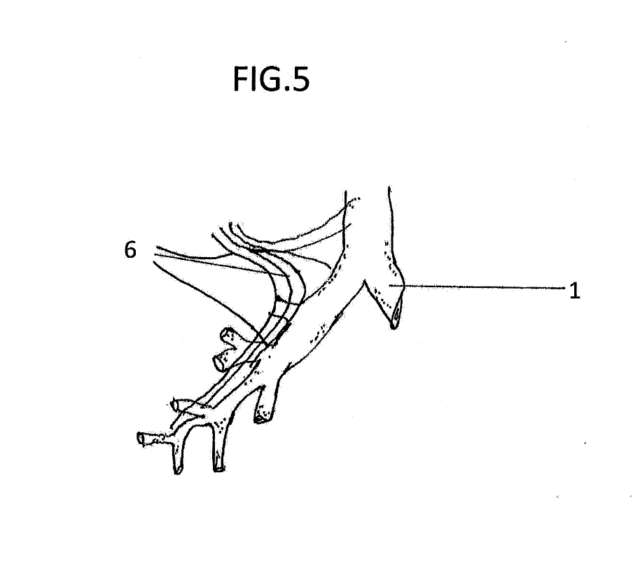

The apparatus also includes a catheter 10 having a proximal end, a distal end and a proximal-to-distal axis which, in the condition depicted in FIG. 4 is preferably coincident with the bronchial axis. Alignment with the bronchial axis will provide for a more homogeneous energy distribution through the treatment volume (see upper diagram in FIG. 4A). In the case of misalignment the energy levels vary greatly from side to side as shown in the lower diagram of FIG. 4B. This will cause wall injury on one side while the other side is ineffective in ablating nerves. Centering will cause the flatter portion of the 1/r curve to determine the energy distribution within the treatment volume as shown in the upper diagram of FIG. 4A. Utilizing the flat portion of the 1/r curve avoids significant energy differentials throughout the treatment volume and reduces the potential for collateral damage, in particular bronchial wall damage. In particular the very high power levels on or close to the emitter surface are positioned inside the bladder volume, where no harm is done since ultrasound does not interact with the cooling fluid.

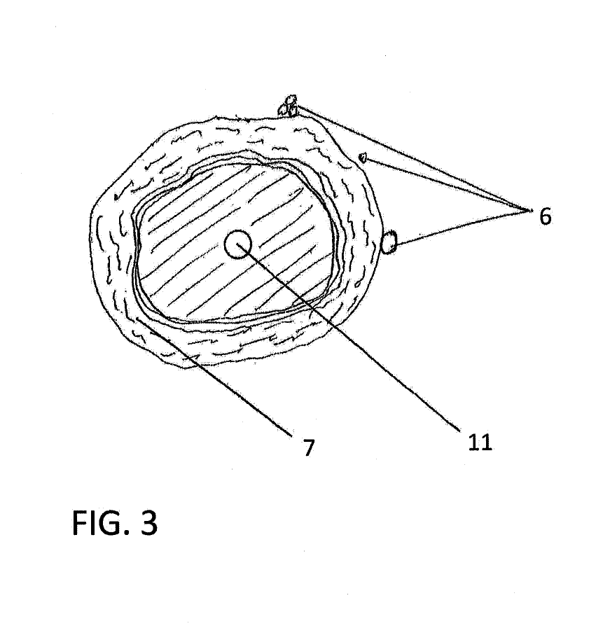

Catheter 10 has a compliant balloon or inflatable bladder 12 mounted at the distal end. In its inflated condition (FIGS. 2 and 3), bladder 12 will engage the bronchial wall and therewith allow for ultrasound to be conducted from transducer into the bronchial wall and surrounding tissues.

An electromechanical transducer 11 (FIG. 3) is mounted adjacent the distal end of catheter 10 within bladder 12. Transducer 11, which is desirably formed from a ceramic piezoelectric material, is of a tubular shape and has an exterior emitting surface. The transducer 11 typically has an axial length of approximately 2-10 mm, and preferably 5 mm. The outer diameter of the transducer 30 is approximately 1.5-3 mm in diameter, and preferably 2 mm. The transducer 11 also has conductive coatings (not shown) on its interior and exterior surfaces. Thus, the transducer may be physically mounted on a metallic support tube which in turn is mounted to the catheter. The coatings are electrically connected to ground and signal wires. Wires extend from the transducer 11 through a lumen in the catheter shaft to a connector electrically coupled with the ultrasound system. The lumen extends between the proximal end and the distal end of a catheter 10, while the wires extend from the transducer 11, through the lumen, to the proximal end of the catheter 10.

Transducer 11 is constructed so that ultrasonic mechanical waveform energy is generated by the transducer and is emitted principally from the exterior and interior surface. In order to increase efficiency, the transducer may include features arranged to reflect ultrasonic energy directed toward the interior of the transducer so that the reflected energy reinforces the ultrasonic vibrations at the exterior surface. For example, support tube and transducer may be configured so that the energy emitted from the interior surface of the transducer 11 is reflected back to enhance the overall efficiency of the transducer. In this embodiment, the ultrasonic mechanical vibratory energy generated by the transducer 11 is reflected at the interior mounting to reinforce ultrasonic mechanical vibratory energy propagating from the exterior surface of the transducer 11.

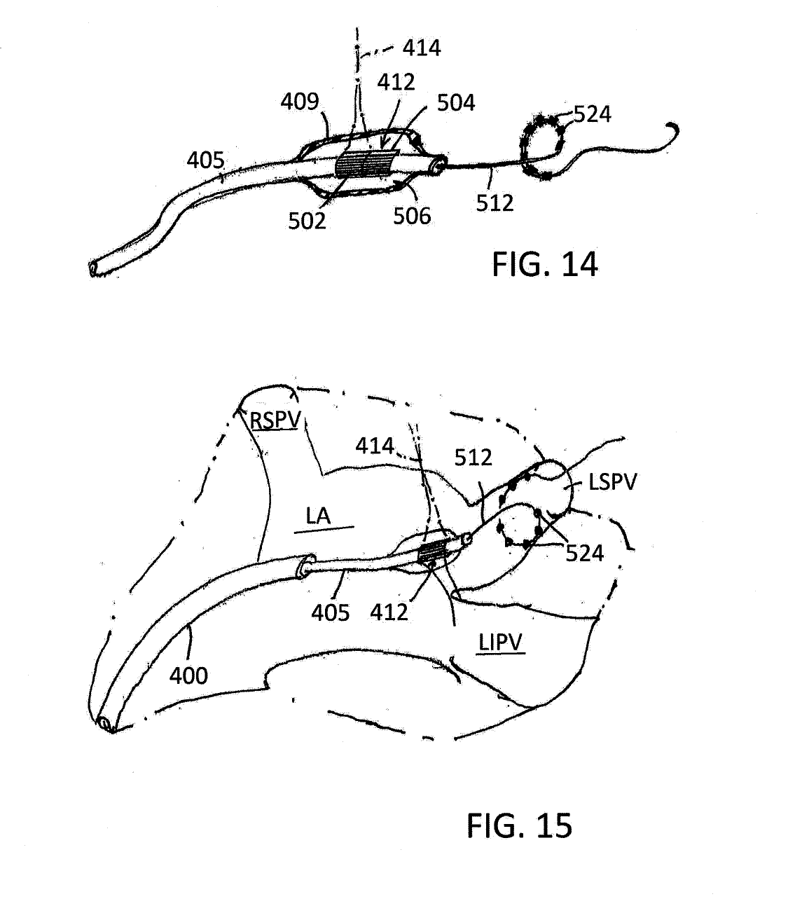

Transducer 11 is also arranged to convert ultrasonic waves vibrating the exterior surface into electrical signals which can be detected by the ultrasound detection subsystem. If the reflecting structure is not perfectly circular the widths of the reflected signal will represent the difference between a maximum internal diameter dₘₐₓ and a minimum internal diameter dₘᵢₙ of the bronchial passageway under treatment. Stated another way, transducer 11 can act either as an ultrasonic emitter or an ultrasonic receiver. The receiving mode is of particular importance for an array type transducer, as described hereinafter with reference to FIGS. 14-18, because with an array type transducer 11 the received echoes can be electronically focused and high resolution images can be achieved.

The transducer 11 is designed to operate, for example, at a frequency of approximately 1 MHz to approximately a few tens of MHz, and typically at approximately 15 MHz given the shallow location of bronchial smooth muscle and nerves. The actual frequency of the transducer 11 typically varies somewhat depending on manufacturing tolerances. The optimum actuation frequency maybe adjusted accordingly by the generator system based on a digital memory, bar code or the like affixed to the catheter.

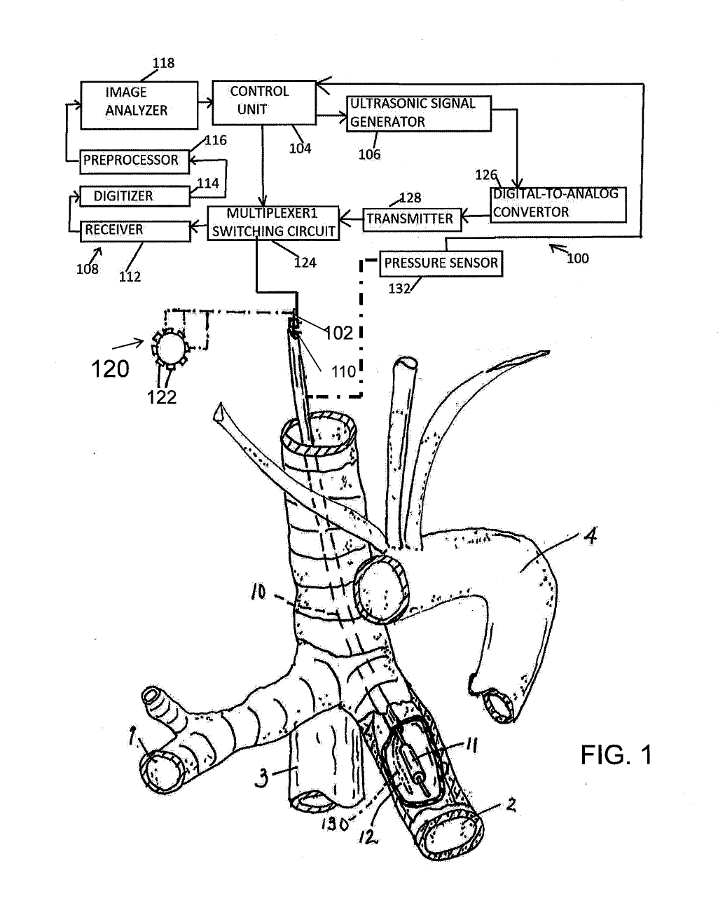

An ultrasound system, also referred to herein as an energization circuit 100 (FIG. 1), is releasably connected to catheter 10 and transducer 11 through a plug connector 102. A control unit 104 and an ultrasonic-signal generator 106 are arranged to control the amplitude and timing of the electrical signals so as to control the power level and duration of the ultrasound signals emitted by transducer 11. The energization circuit 100 also includes a detection subcircuit 108 arranged to detect electrical signals generated by transducer 11 and appearing on wires 110 and communicate such signals to the control unit 104. More particularly, detection subcircuit 108 includes a receiver or echo signal extractor 112, a digitizer 114, an ultrasonic echo signal preprocessor 116, and an image analyzer 118 connected in series to one another. Ultrasonic signal generator 106 produces both therapeutic denervation signals and outgoing diagnostic imaging signals. As discussed hereinafter, the outgoing imaging signals and the returning echo signals may be transmitted and picked up by a circular array 120 of transducer elements 122 operating as a phased array. A multiplexer or switching circuit 124 is operated by control unit 104 to switch to a receiving mode after imaging signals are emitted during a transmitting mode via a digital-to-analog converter 126 and a transmitter module 128.

A circulation device is connected to lumens (not shown) within catheter 10 which in turn are connected to bladder 12. The circulation device is arranged to circulate a liquid, preferably a sterile aqueous liquid, through the catheter 10 to the transducer 11 in the bladder 12. The circulation device may include elements for holding the circulating coolant, pumps, a refrigerating coil (not shown), for providing a supply of liquid to the interior space of the bladder 12 at a controlled temperature, desirably at or below body temperature. The control board interfaces with the circulation device to control the flow of fluid into and out of the bladder 12. For example, the control board may include motor control devices linked to drive motors associated with pumps for controlling the speed of operation of the pumps. Such motor control devices can be used, for example, where the pumps are positive displacement pumps, such as peristaltic pumps. Alternatively or additionally, the control circuit may include structures such as controllable valves connected in the fluid circuit for varying resistance of the circuit to fluid flow (not shown). The ultrasound system may further include pressure sensors, to monitor the liquid flow through the catheter 10. At least one pressure sensor monitors the flow of the liquid to the distal end of catheter 10 to determine if there is a blockage while the other monitors leaks in the catheter 10. While the balloon is in an inflated state, the pressure sensors maintain a desired pressure in the balloon preferably so that the compliant balloon occludes the bronchus.

The ultrasound system incorporates a reader for reading a machine-readable element on catheter 10 and conveying the information from such element to the control board. As discussed above, the machine-readable element on the catheter may include information such as the operating frequency and efficiency of the transducer 11 in a particular catheter 10, and the control board may use this information to set the appropriate frequency and power for exciting the transducer. Alternatively, the control board may be arranged to actuate an excitation source to measure the transducer operating frequency by energizing the transducer at a low power level while scanning the excitation frequency over a pre-determined range of frequencies for example 5.0 Mhz-15.0 Mhz, and monitoring the response of the transducer to such excitation and to select the optimal operating frequency.

The ultrasonic system may be similar to that disclosed hereinafter with reference to FIGS. 14-18.

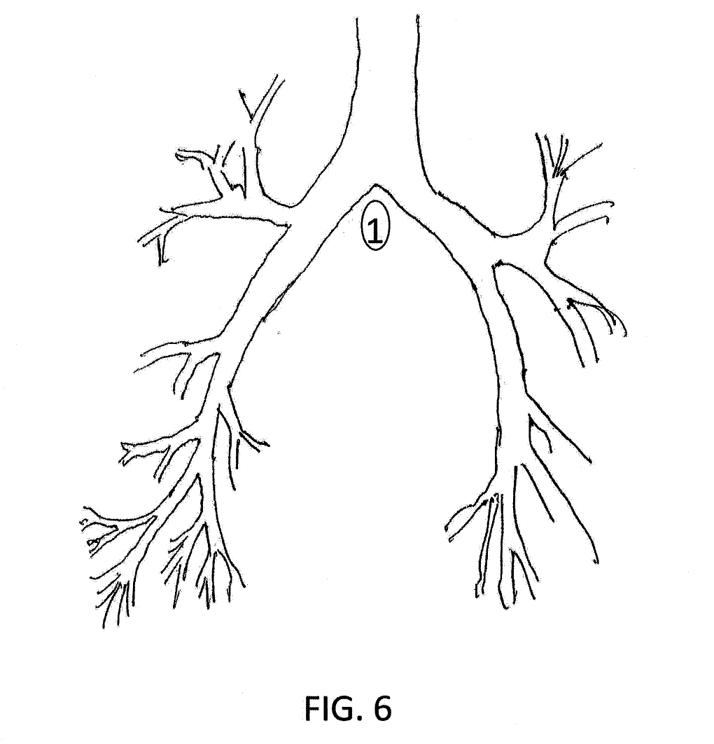

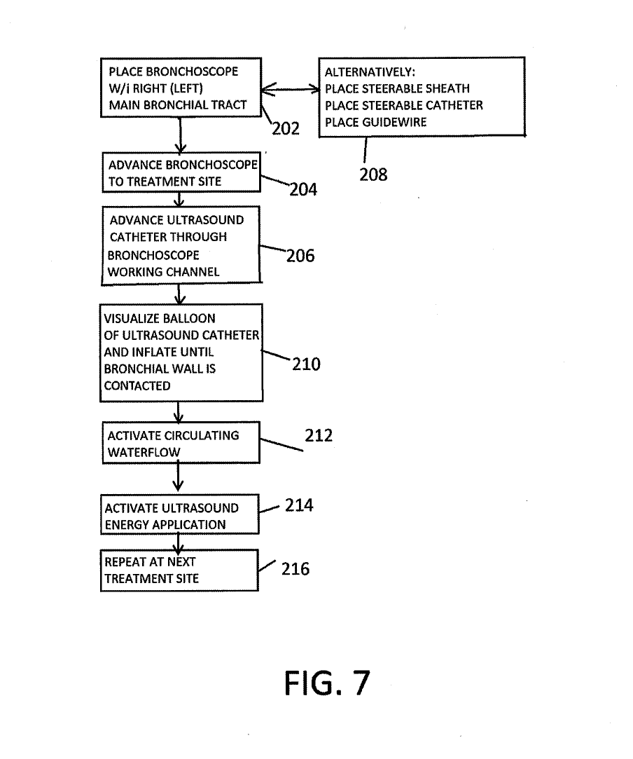

A method according to an embodiment of the present invention is depicted in flowchart form in FIG. 7. After preparing the tracheal access site of a human or non-human mammalian subject such as a patient, and connecting the catheter 10 to the ultrasound system, the ultrasound catheter is inserted into the working channel of the bronchoscope (step 206) after the bronchoscope has been advanced (steps 202 and 204) to the desired treatment site under visual guidance via the bronchoscope camera or optical fiber. Alternatively, a steerable sheath, preferably with ultrasound imaging capability as described hereinafter with reference to FIGS. 8-13, can be used as a delivery channel for the treatment catheter (step 208). In another embodiment the treatment catheter is equipped with a steering or deflection mechanism and can be advanced directly to the treatment site as shown in FIG. 1. If the catheter combines imaging and therapeutic capabilities as described in the '818 provisional application, this delivery method enables the fastest procedure time and is easily tolerated by the patient. Yet another embodiment provides for a guide wire 14 (in FIG. 2) to be delivered through the working channel of the bronchoscope to the treatment site and the ultrasound treatment catheter to be advanced over the wire after the bronchoscope has been withdrawn. This technique will allow for very small, flexible bronchoscopes to be utilized. In another embodiment an optical fiber 130 (FIG. 1) is inserted through the central catheter lumen to allow for optical guidance during catheter insertion and manipulation.

Once the distal end of the catheter is in position within a bronchial branch, pumps bring bladder 12 to an inflated condition (steps 210 and 212 in FIG. 7) as depicted in FIGS. 2 and 3. In this condition, the compliant bladder 12 engages the bronchial wall, and thus centers transducer 11 within the bronchial branch, with the axis of the transducer approximately coaxial with the axis of the bronchial branch. This not only provides for a relatively homogeneous energy distribution circumferentially, but also keeps the very high energy levels close to the transducer located inside the cooling fluid where they are harmless, since ultrasound does not interact with fluid (see FIG. 4). If these peak energy levels where allowed to be located close to the bronchial wall (1), injury would result. These two situations are shown in FIG. 4 where in the upper drawing 4A the electromechanical transducer is properly centered and the energy is distributed without causing wall (1) injury. The other advantage of proper centering is that the treatment volume is coinciding with the relatively flat portion of the 1/r curve providing an almost constant power level throughout the treatment volume. In the lower drawing 4B of FIG. 4, the transducer is not centered, resulting in uneven power distribution circumferentially. Also, the transducer is positioned off axis (due to too small a balloon diameter) which exposes the bronchial wall to a peak power level which may cause wall injury.

During treatment with ultrasonic vibrational energy (step 214 in FIG. 7), the circulation device maintains a flow of cooled aqueous liquid into and out of bladder 12, so as to cool the transducer 11 (step 212). The cooled balloon also tends to cool the interior surface of the bronchus. The liquid flowing within the balloon may include a radiographic contrast agent to aid in visualization of the balloon and verification of proper placement.

In another embodiment, the ultrasound system uses transducer 11 to measure the size of the bronchus. The control board and ultrasound source actuate the transducer 11 to emit short, low power signals which will be reflected by the bronchus. The ultrasonic waves in this pulse are reflected by the bronchial wall onto transducer 11 as echoes. Transducer 11 converts the echoes to echo signals. The ultrasound system then determines the size of bronchus 1 by analyzing the echo signals. For example, the ultrasound system may determine the time delay between actuation of the transducer and reception of the echoes representing the bronchial radius. The width of the return signal or echo represents the difference between dₘₐₓ and dₘᵢₙ in case the bronchial section is not perfectly circular but oval shaped. The ultrasound system uses the measured bronchus size to set the acoustic power to be delivered by transducer 11 during application of therapeutic ultrasonic energy in later steps. For example, the control board may use a lookup table correlating a particular echo delay (and thus bronchial radius) with a particular power level. Generally, the larger the diameter, the more power should be used.

The physician then initiates the treatment through the user interface. In the treatment, the ultrasonic signal generating system or energization circuit, and particularly the control board and ultrasonic source, actuate transducer 11 to deliver therapeutically effective ultrasonic waves to an target or ultrasound treatment region 13 (FIG. 2). The ultrasonic mechanical vibratory energy transmitted by the transducer 11 propagates generally radially outwardly and away from the transducer 11 encompassing a full circle, or 360° of arc about the proximal-to-distal axis of the transducer 11 and the axis of the bronchial section treated.

The selected operating frequency, unfocused characteristic, placement, size, and the shape of the electromechanical transducer 11 allows the entire bronchial section and bronchial nerves to lie within the “near field” region of the transducer 11. As shown in FIG. 2 within this region, an outwardly spreading, unfocused (360°) cylindrical beam of ultrasound waves generated by the transducer 11 tends to remain collimated. For a cylindrical transducer, the radial extent of the near field region is defined by the expression L²/λ, where L is the axial length of the transducer 11 and λ is the wavelength of the ultrasound waves. At distances from the transducer 11 surface greater than L²/λ, the beam begins to spread axially to a substantial extent. However, for distances less than L²/λ, the beam does not spread axially to any substantial extent (FIG. 2). Therefore, within the near field region, at distances less than L²/λ, the intensity of the ultrasonic mechanical vibratory energy decreases according 1/r as the unfocused beam spreads radially. As used in this disclosure, the term “unfocused” refers to a beam, which does not increase in intensity in the direction of propagation of the beam away from the transducer 11.

The target region 13 is generally cylindrical and coaxial with the bronchial section treated (FIG. 2). It extends from the transducer surface to an impact radius, where the intensity of the ultrasonic energy is too small to heat the tissue to the temperature range that will cause inactivation of nerves.

As discussed above, the length of the transducer 11 may vary between about 2 mm and about 10 mm, but is preferably about 5 mm to provide a wide inactivation zone of the bronchial nerves. The diameter of the transducer 11 may vary between 1.5 mm to 3.0 mm, and is preferably less than 2.0 mm in order to allow the catheter to fit through the bronchoscope working channel. The dosage is selected not only for its therapeutic effect, but also to allow the radius of the target region 13 to be between preferably 5 mm and 10 mm in order to encompass the bronchial section treated, and adjacent bronchial nerves, all of which lie within an average radius of 5-10 mm, without transmitting damaging ultrasonic mechanical vibratory energy to collateral structures like esophagus 3 and Aorta 4 in FIG. 1.

The power level desirably is selected so that throughout the target region, solid tissues are heated to about 42° C. or more for several seconds or more, but desirably all of the solid tissues, including the wall of the bronchus remain well below 65° C. Thus, throughout the impact region, the solid tissues (including all of the bronchial nerves) are brought to a temperature sufficient to inactivate nerve conduction but below that which causes rapid necrosis of the tissues.

Research shows that nerve damage occurs at much lower temperatures and much faster than tissue necrosis. See Bunch, Jared. T. et al. “Mechanisms of Phrenic Nerve Injury During Radiofrequency Ablation at the Pulmonary Vein Orifice, Journal of Cardiovascular Electrophysiology, Volume 16, Issue 12, pg. 1318-1325 (Dec. 8, 2005), incorporated by reference herein. Since, necrosis of tissue typically occurs at temperatures of 65° C. or higher for approximately 10 sec or longer while inactivation of nerves typically occurs when the nerves are at temperatures of 42° C. or higher for several seconds or longer, the dosage of the ultrasonic mechanical vibratory energy is chosen to keep the temperature in the target region 13 between those temperatures for several seconds or longer. In addition, the circulation of cooled liquid through the bladder 12 containing the transducer 11 may also help reduce the heat being transferred from the transducer 11 to the inner layer of the bronchus. Hence, the transmitted therapeutic unfocused ultrasonic mechanical vibratory energy does not damage the inner layer of the bronchus, providing a safer treatment.

In order to generate the therapeutic dosage of ultrasonic mechanical vibratory energy, the acoustic power output of the transducer 11 typically is approximately 10 watts to approximately 100 watts, more typically approximately 10 watts. The duration of power application typically is approximately 2 seconds to approximately a minute or more, more typically approximately 10 seconds. The optimum dosage used with a particular system to achieve the desired temperature levels has been determined by mathematical modeling and animal testing to be 100 Joules for a 5 mm bronchial lumen.

The target region 13 of the unfocused ultrasonic mechanical vibratory energy encompasses the entire bronchial section treated and closely surrounding tissues, and therefore ablates all of the bronchial nerves surrounding the bronchus. Accordingly, the placement in the bronchus of the transducer 11 may be indiscriminate in order to inactivate conduction of all the surrounding bronchial nerves 6 surrounding the bronchi in the subject.

Optionally, the physician may then reposition the catheter 10 and transducer 11 along the bronchus and reinitiate the treatment to retransmit therapeutically effective unfocused ultrasonic mechanical vibratory energy. This inactivates the bronchial nerves at an additional location along the length of the bronchial tree, and thus provides a safer and more reliable treatment. The repositioning and retransmission steps optionally can be performed multiple times. Next the physician moves the catheter 10 with the transducer 11 to the other main bronchus (le/ri) and performs the entire treatment again (step 216, FIG. 7) for that bronchial side (see FIG. 6). After completion of the treatment, the catheter 10 is withdrawn from the subject's lungs.

Numerous variations and combinations of the features discussed above can be utilized. For example, the ultrasound system may control the transducer 11 to transmit ultrasonic mechanical vibratory energy in a pulsed function during application of therapeutic ultrasonic energy. The pulsed function causes the electromechanical transducer 11 to emit the ultrasonic mechanical vibratory energy at a duty cycle of, for example, 50%. Pulse modulation of the ultrasonic mechanical vibratory energy is helpful in limiting the tissue temperature while increasing treatment times. The pulsed therapeutic function can also be interleaved with a diagnostic imaging mode when an ultrasound array is used instead of a cylindrical solid transducer. This way diagnostic ultrasound imaging can be obtained (quasi)simultaneously to the therapeutic treatment.

In a further variant, the bronchial diameters can be measured by techniques other than actuation of transducer as, for example, by radiographic imaging or magnetic resonance imaging or use of a separate ultrasonic measuring catheter. In this instance, the data from the separate measurement can be used to set the dose.

Bladder 12 is typically cylindrical, that is, it has a circular cross-section and a cylindrical outer surface which makes contact with the wall of the targeted bronchial section. Where the inner surface of the bronchial section being treated is non-circular, the balloon may deform under liquid pressure to conform to the bronchial surface. Ultrasound transmissibility between the bladder and the bronchial wall may be enhanced by providing the outer surface of the bladder with a layer of liquid, for instance, saline solution or biocompatible gel. This is especially advantageous if the bronchial wall is not already coated with mucous or other fluidic material. The layer of liquid on the outer surface of bladder 12 may be provided during the manufacturing process or may be provided at the time of the therapeutic treatment. In the latter case, the liquid may be sprayed onto the bladder inside the bronchial passage, using a catheter with a spray nozzle. The liquid may be provided via catheter 10, in which case the catheter is connected at a proximal end to a source of pressurized liquid.

Typically, catheter 10 is a disposable, single-use device. The catheter 10 or ultrasonic system may contain a safety device that inhibits the reuse of the catheter 10 after a single use. Such safety devices per se are known in the art.

In yet another variant, the catheter 10 itself may include a steering mechanism which allows the physician to directly steer the distal end of the catheter. In this case a bronchoscope or sheath may be omitted. Of particular advantage in this mode is insertion of an optical fiber (e.g., 130 in FIG. 1) through the central catheter lumen for optical guidance during catheter insertion and manipulation.

Another variation may be that an ultrasonic waveform emitter unit at the distal end of the catheter, which includes the electromechanical transducer, may be positioned in adjacent structures like the pulmonary artery or the esophagus (3 in FIG. 1), and the electromechanical transducer may include reflective or blocking structures for selectively directing ultrasonic mechanical vibratory energy from the transducer over only a limited range of radial directions to provide that ultrasonic mechanical vibratory energy desirably is selectively directed from the transducer in the adjacent structure toward the bronchial nerves. When this approach is utilized, the ultrasonic mechanical vibratory energy is directed into a segment or beam propagating away from an exterior surface of the transducer, commonly known as a side firing transducer arrangement. For example, the electromechanical transducer may have a construction and be operated to emit as an ultrasound array and directed ultrasonic mechanical vibratory energy under image guidance similarly as disclosed herein. In this variation, the route by which the catheter is introduced into the body, and then positioned close to the bronchus, is varied from the bronchial approach discussed above.

Imaging apparatus useful in methods disclosed hereinabove includes a sheath 301 (FIG. 8) generally in the form of an elongated tube having a proximal end 320, a distal end 330 and a proximal-to-distal axis. As used in this disclosure with reference to elongated elements for insertion into the body, the term “distal” refers to the end which is inserted into the body first, i.e., the leading end during advancement of the element into the body, whereas the term “proximal” refers to the opposite end.

Sheath 301 has an interior bore or lumen (not separately designated) extending between its proximal end 320 and its distal end 330. Desirably, sheath 301 has a relatively stiff proximal wall section 341 extending from its proximal end 320 to a juncture 340, and a relatively soft distal wall section or sheath end portion 342 extending from the juncture 340 to the distal end or tip 330. One or more pull wires 344 (only one shown) are slideably mounted in the proximal wall section 341 and connected to the distal wall section or end portion 342. The pull wire 344 is linked to a pull wire control apparatus (not shown), which can be manipulated by a physician during use of the sheath 301. The structure of sheath 301 and pull wire control may be generally as shown in U.S. Patent Application Publication No. 2006-0270976 (“the '976 Publication”), the disclosure of which is incorporated by reference herein. As discussed in greater detail in the '976 Publication, transition desirably is oblique to the proximal-to-distal axis 346 of the sheath.

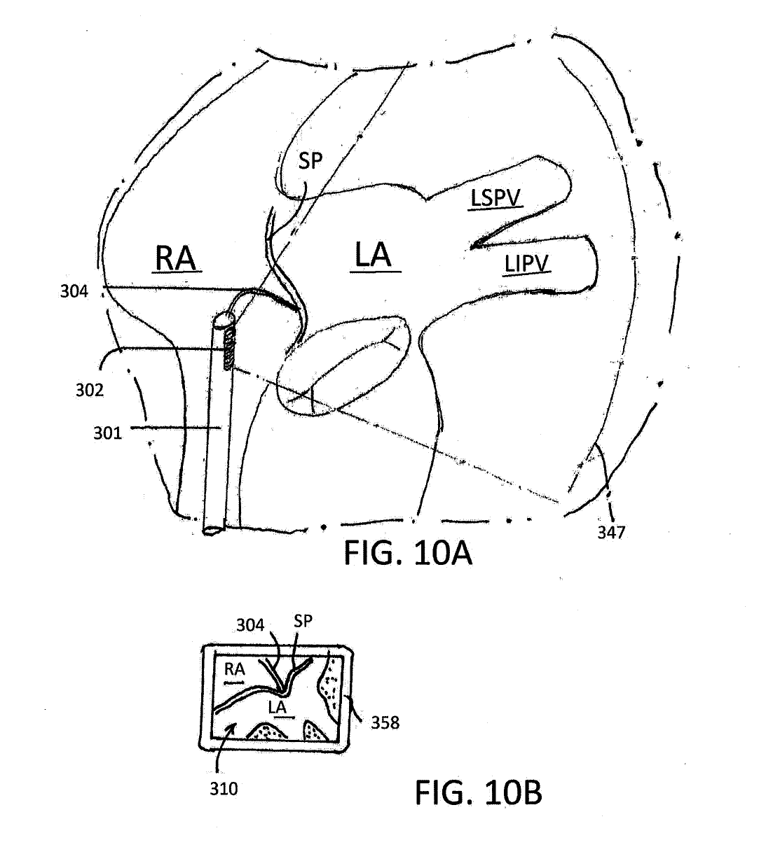

By combined pulling on the pull wire 344 and rotational motion, the distal end 330 of sheath 301 and therewith an ultrasound imaging plane 347 (FIGS. 9A, 10A) can be aimed in essentially any desired direction. As disclosed in the aforementioned '976 Publication, the pull wire control can be incorporated into a handle which is physically attached to the proximal end 320 of the sheath 301. Thus, the physician can maneuver the sheath 301 by actuating the pull wire control and turning the handle, desirably with one hand, during the procedure.

The apparatus further includes, in the distal wall section or sheath end portion 342, a circular array 302 of electromechanical (e.g., PZT or piezoelectric) transducer elements for ultrasound imaging. As described above, the sheath steering allows the physician to aim the sheath distal opening (at 330) in any direction and through the same steering operation to aim the ultrasound imaging plane 347 in any direction.

In order to keep the sheath wall reasonably thin printed flexible circuits 311 (see FIG. 12) are employed to electrically connect the ultrasound transducer array 302 with one or more multiplexer integrated circuits (ICs) 312. In one embodiment this flex circuit 311 can be an outermost sheath layer dimensioned to act as a lambda/4 impedance matching layer. The acoustic impedance of this matching layer is selected to optimize the acoustic transition from the semiconductor material of the ultrasound transducers of array 302 to body tissue or blood: Z_{match}=SQRT(Z_{PZT}×Z_{Blood}). Preferably, several matching layers are provided. In this embodiment the ultrasound array 302, which can consist of PZT, is mounted with a die attach film 348 onto the flex circuit 311. The material of die attach film 348 (e.g., Henkel CF3350) and the thickness thereof are chosen so that the film acts as a second matching layer: Z_{MatchFilm}=SQRT(Z_{pzt}×Z_{flex}) and Z_{MatchFlex}=SQRT(Z_{film}×Z_{blood}). In an alternative embodiment the electronic circuitry is printed onto the innermost, extruded, sheath layer and then covered isometrically with an outer sheath layer which acts as one or one of several matching layers.

Another desirable feature of the present imaging sheaths is to keep the overall diameter isometric (no bulge).

In order to keep the sheath wall reasonably thin the number of connections with the ultrasound imaging console has to be minimized. Therefore a multiplexer approach is employed: with two 64:16 multiplexers 12 as shown in FIG. 12, 128 transducer elements of array 302 can be controlled with 2×16 signal lines plus supply voltage and control lines 313 running within the sheath wall from proximal end 320 to the distal end portion 342. For 3D imaging 2-dimensional arrays are required and several (n) multiplexers are employed to reduce the high array element numbers by n×64 (in case of 64:16 multiplexers).

At the proximal end the lines are terminated in a connector 352 (FIG. 12) which is mated with a connector cable 354 from a control unit 356 which feeds a video signal to an imaging console or display 358. This connector cable 352 is supplied sterile and one end placed by the sterile operator in the sterile field (to be connected to the imaging sheath) while the other end is connected to the system in the non-sterile field.

Particular attention has to be paid to the backing of array 302. For imaging purposes highly absorptive backing is desirable. This contradicts with the size requirements to keep the sheath wall acceptably thin. Accordingly, minimal backing is applied to array 302 of sheath 301. Rather than absorbing the backwards emitted ultrasound portion a diffraction layer 360 is employed to cause the backward-propagating ultrasound waves to bounce back and forth in chaotic fashion within the blood filled sheath 301. This way the backwardly emitted ultrasound is prevented from generating reverberations within the ultrasound image. Diffraction layer may be made of polyimide with a conductive layer, for example, Pyralux from DuPont.

A further variation of an combined imaging/therapy sheath, depicted in FIG. 13, includes a tubular member 361 provided with a split transducer array 364, where one circular or annular section 362 is optimized for imaging with the above described diffraction mechanism (layer 360) and another circular or annular section 368 optimized for therapy. The therapy section 368 employs a metallic backing 370 to reflect a backward-propagating ultrasound wave front forward. Preferably the reflector backing 370 is spaced by a water-filled gap or distance 371 of lambda/2 behind an inner or rear surface of the transducer section 368. FIG. 13 also depicts electrodes 372, 374 sandwiching a piezoelectric or PZT layer 376, a die attach film 378, and flex circuit layer 380 in the imaging transducer section 362, with an analogous structure being present in the therapy transducer section 368. The split array configuration is described in further detail hereinafter.

Numerous other variations and combinations of the features discussed above can be utilized. For example, the emitter structure can be slideably mounted within the sheath so that the sheath stays in place during the procedure. In still other arrangements, several emitters might be mounted on the sheath in a chain like fashion in order to apply energy over the length of the sheath portion inserted into the organ to be treated. Again this configuration does not require a movement of the sheath during treatment. In still other embodiments, focusing apparatus, such as lenses and diffractive elements can be employed in particular for short axis focusing of the ultrasonic energy. The right atrial position in case of intra cardiac procedures allows the user to obtain real time guidance of the trans-septal puncture as well as the left atrial catheter ablation itself.

The right atrial sheath position in case of intra cardiac procedures allows the user to obtain real time guidance of the trans-septal puncture as well as the catheter ablation itself. As depicted in FIG. 9A, sheath 301 is percutaneously inserted into the venous vascular system of a patient so that the distal wall section or sheath end portion 342 is disposed in the patient's right atrium RA. Sheath 301 carries circumferential imaging array 302. A Brockenbrough needle 304 is advanced through sheath 301 under ultrasound imaging guidance to puncture the septum SP. The user will observe the tenting effect of the needle 304 on the septum SP in the ultrasound image 310 on display 358 (FIG. 9B). This will allow the user to choose an optimal puncture site and reduce the chances for collateral damage.

FIG. 10A shows a variation of the procedure of FIG. 9A, with a sheath 372 having a longitudinal ultrasound imaging array 374. FIG. 10B shows an associated ultrasound-obtained image 310 on display 358.

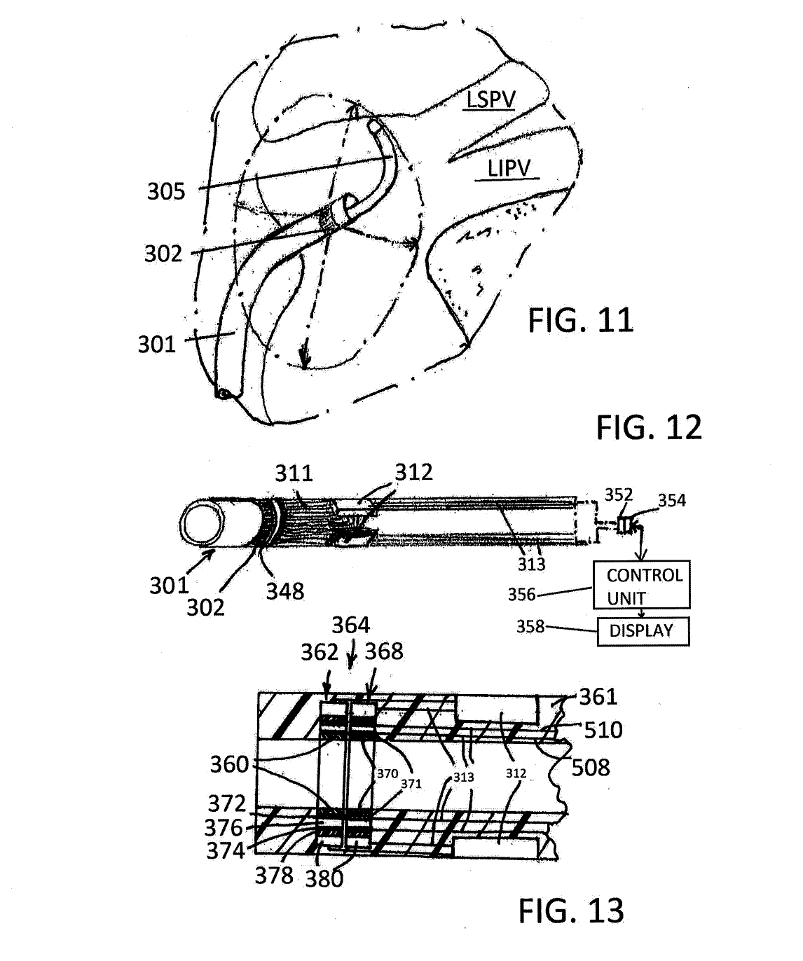

All left sided cardiac interventions require a trans-septal puncture to be performed. As described above ultrasound guidance has great value since tenting of the septum clearly indicates the puncture site. Once the septum has been crossed the imaging sheath 301 can be advanced into the left atrium LA to guide the therapeutic procedure. In case of an AF treatment procedure, a distal end portion (not separately enumerated) of an ablation catheter 305 is ejected from sheath 301 and maneuvered into a pulmonary vein, e.g., left superior pulmonary vein LSPV, as shown in FIG. 11.

FIG. 14 illustrates related catheter-based composite imaging and therapy apparatus adapted for performing a pulmonary vein isolation procedure in treatment of atrial fibrillation. The same or similar apparatus can be used for forming annular ablations along inner surfaces of other tubular or hollow organs such as the urinary tract, the esophagus and bronchial tubes.

An expansible structure in the form of a balloon 409 (FIG. 14) is mounted to a distal end of a catheter 405. In the inflated, operative condition the balloon 409 provides a water/contrast filled volume to cool an energy emitter in case of ultrasound energy and to make it easily visible in fluoroscopy.

A tubular, cylindrical ultrasonic transducer array 412 is mounted to catheter 405 inside balloon 409. Transducer array 412 includes a plurality of electrically isolated and independently energizable piezoelectric or PZT transducer elements organized into a therapy transducer section 502 and an imaging transducer section 504 (FIG. 14). Therapy transducer section 502 is backed either with air or at a lamda/2 distance with a metal reflector (370, FIG. 13) in water to reflect most ultrasound energy forward or outwardly into an active beam segment 414 which will overlap with the antrum of a PV annulus section being treated. In case of a reflector the space between the piezoelectric or PZT transducer elements and the reflector communicates with an interior cooling fluid filled space 506 within balloon 409 which provides additional cooling for the transducer 412. Metallic coatings (see 372, 374, FIG. 13) on the interior and exterior surfaces of the array elements (or front and back in case of a planar design) serve as excitation electrodes and are connected to a ground wire 508 and a signal wire 510 which extend through a wiring support tube to the distal end of the catheter. The wires 508 and 510 are connected to an ultrasonic excitation source 415 (FIG. 10) and a console or monitor 513 of an ultrasound imaging system. The process of forming such cylindrical arrays is well known and described in the prior art, see Eberle U.S. Pat. No. 6,049,958.

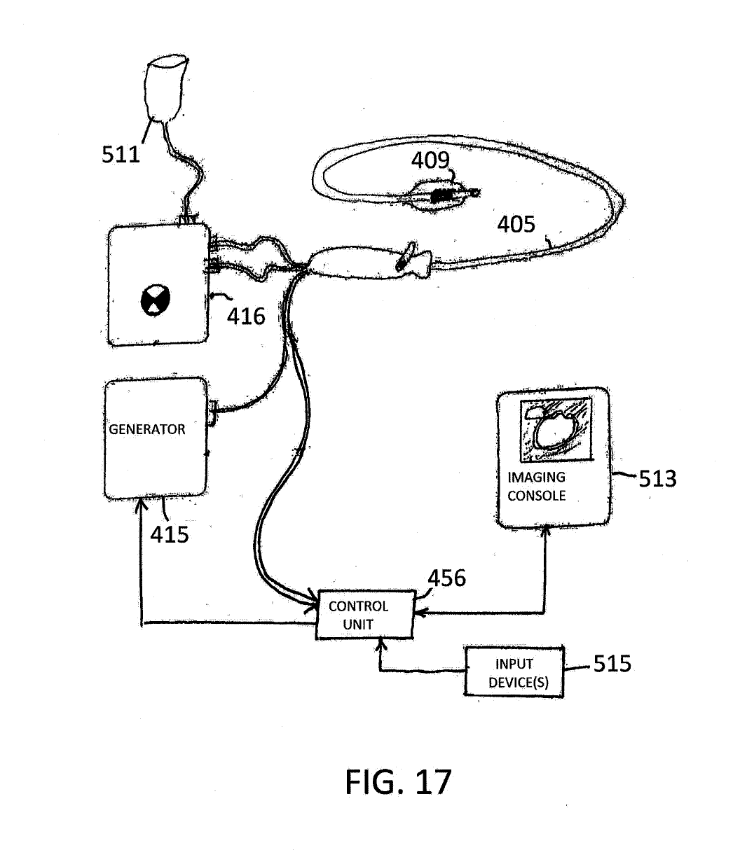

Electrical connection of the piezoelectric elements of array 412 with generator 415 and an imaging display or monitor 513 of a control system 456 (FIG. 17) is best achieved through flex circuit strip lines. In order to reduce the line count, multiplexer IC's can be deployed at the distal end of catheter 105, preferably close to ultrasound array 412. (See 312, FIGS. 12 and 13.) Of advantage are multiplexer circuits directly deposited at the distal end of the strip lines in a staggered fashion to keep the catheter diameter small.

The interior space 506 within balloon 409 is connected to a circulation device 416 (FIG. 17) for circulating a liquid, preferably an aqueous liquid, from a liquid source or supply 511 through the balloon to cool the ultrasound transducer 412 in order to avoid blood coagulation. Circulation device 416 includes at least one pump. As further discussed below, during operation, the circulation device 416 continually circulates the aqueous fluid through the balloon 409 and maintains the balloon under a desired pressure and temperature.

Catheter 405 is deployed via a sheath 400 (FIG. 15) generally in the form of an elongated tube having a proximal end, a distal end and a proximal-to-distal axis. Sheath 400 is advanced over a guide-wire through femoral access into the right atrium. After a septal puncture has been performed the catheter 405 is advanced through the sheath 400 into the left atrium LA (FIG. 15).

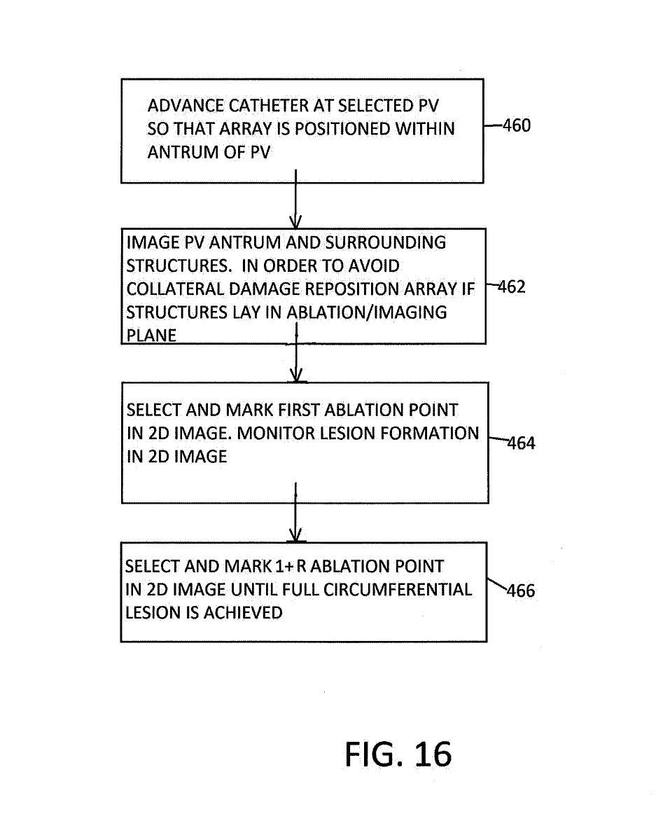

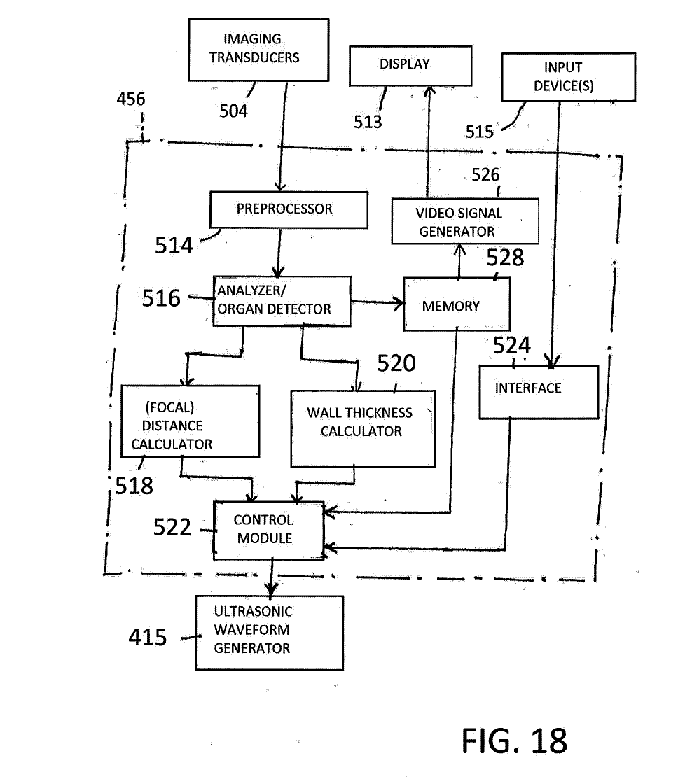

Treatment catheter 405 is advanced under ultrasound image guidance until the antrum of the selected pulmonary vein (PV) is clearly visualized. Treatment catheter is advanced further so that ultrasound transducer array 412 is positioned within the antrum of a selected pulmonary vein (PV) (step 460, FIG. 16). Ultrasound imaging guidance will reduce the need for fluoroscopic imaging and cut down on ionizing radiation. Once the treatment catheter has been positioned and mechanically stabilized by means of a sensing loop catheter 512 the ablation process can be controlled through the imaging system from the control room (steps 462, FIG. 16). Interactively ablation targets are identified in the image with markers (step 464, 466). The markers are instructions input to the control unit 456 (FIG. 18, or 356, FIG. 12), exemplarily via a touch screen (358, 513) or a keyboard and/or mouse input device (515), that indicate the location of a desired ablation on the organic structures represented in the displayed image. As discussed hereinafter in detail with reference to FIG. 18, the control system 456 translates these ablation markers into focusing, power and time parameters to control the ablation beam in the desired location and to ablate a lesion of the appropriate depth. During the ablation process the ablation site is monitored via ultrasound in an interlaced mode to allow the user to control the ablation process under essentially real time visualization. Since ablated tissue increases ultrasound reflectivity an intensity change can be observed during ablation. Ablated tissue clearly shows higher reflectivity than non ablated tissue so that the ablation can be terminated when a transmural lesion has been obtained. This will reduce the potential for collateral damage through over dosing.

With the catheter in the operative position, the energy field 414 (FIG. 14) is aligned with one point of the PV antrum image. In other words the therapy transducer section 502 is set under programming to focus ultrasonic vibration energy on the particular location of the organ to be treated. The imaging transducer section 504 communicates, to the computer system control unit 456, ultrasonic waveform data from which the computer calculates distance of the therapy transducer section 502 from the atrial wall and the thickness of the atrial wall at the particular location of the antrum. More specifically, ultrasonic waveform generator 415 transmits an electrical signal of one or more pre-established ultrasonic frequencies to a selected transmitting transducer element of transducer array 412. Reflected ultrasonic waveform energy from internal organic structures of the patient is detected by sensor transducer elements of imaging transducer section 504 and processed by a preprocessor 514. Preprocessor 514 is connected to a signal analyzer 516 that computes dimensions and shapes of the internal organic structures. Output of analyzer 516 is organized and compared by a distance detector 518 to determine the distance of therapy transducer section 502 from the target location on the antrum or atrial wall, while an organ thickness detector 520 operates to compare echo signals to thereby determine the thickness of the pulmonary vein at the target location. Distance detector 518 and thickness detector 520 are connected to a therapy signal control module 522 that controls signal generator 415 to so energize the piezoelectric or PZT elements of therapy transducer section 502 in a phased array operation mode as to focus ultrasonic mechanical waves on the target location for a limited ablation time and power. Control module 522 may include a calculation submodule for determining the power and duration parameters of each ablation burst of ultrasonic mechanical waveform energy. The user can monitor the lesion formation in the ultrasound image on display console 513 and override the therapy system if so desired.

Control unit 456 includes an interface 524 for monitoring instructions input by the user via touch screen (360, 513) or keyboard and mouse (515). Signal analyzer 516 is connected to an image signal generator 526 that produces a video signal for display console 513 (or 360) and interface 524 is connected to control module 522 which interprets user directions in conjunction with the organic structures of the patient as detected, encoded and at least temporarily stored in memory 528 by analyzer 516.

As indicated above, ablation is performed preferably in stepwise fashion around a circumferential locus defined by the user or surgeon via the input ablation markers. A neighboring ablation position is chosen as indicated in FIG. 16 and so on until a circumferential, continuous lesion has been created.

With the treatment catheter 405 and transducer array 412 in the operative position, the ultrasonic excitation source or waveform generator 415 actuates the therapy transducer section 502 of transducer array 412 to emit ultrasonic waves. Merely by way of example, the ultrasonic ablation waves (which are longitudinal compression waves) may have a frequency of about 1 MHz to a few tens of MHz, most typically about 8 MHz. The transducer typically is driven to emit, for example, about 10 watts to about 100 watts of acoustic power, most typically about 40 to 50 watts. The actuation is continued for about 10 seconds to about a minute or more, most typically about 20 seconds to about 40 seconds per lesion. Optionally, based on the ultrasound image the actuation may be repeated several times. The frequencies, power levels, and actuation times may be varied from those given above.

The various components of control unit 456 may be hard wired circuits designed to perform the specific computations discussed herein. Alternatively, control unit 456 may take the form of a generic microprocessor or computer with the components realized as generic digital circuits modified by programming to carry out the delineated functions.

The ultrasonic waves generated by the transducer array 412 propagate generally radially outwardly from the transducer elements, outwardly through the liquid within the balloon 409 to the wall of the balloon and then to the surrounding blood and tissue. The ultrasonic waves impinge on the tissues of the heart particularly on the PV antrum. Because the liquid within the balloon and the blood surrounding the balloon have approximately the same acoustic impedance, there is little or no reflection of ultrasonic waves at interfaces between the liquid within the balloon 409 and the blood outside the balloon.

Essentially all of the annulus within the PV antrum lies within the “near field” region of the transducer and particularly the therapy transducer section 502. Within this region, the outwardly spreading segmental beam 414 of ultrasonic waves tends to remain focused not only in the cross-sectional plane but also in elevation axis and has an axial length (the dimension of the beam along the catheter axis; see drawings in FIGS. 8 and 9) roughly equal to the axial length of the transducer section 502 for frequencies of a few MHz in body tissue.

The ultrasonic energy applied by the therapy transducer section 502 is effective to heat and thus necrose a section of the annulus in the PV antrum. A circular lesion formed by a continuous series of sectional ablations creates a conduction block which may be confirmed through lack of PV potentials detected with the loop sensing catheter 512. (Catheter 512 carries a series of mutually spaced sensing electrodes 524 that detect voltage potentials in the cardiac tissue.) The circumferential lesion may take on a variety of shapes (oval or more complicated shapes) and depends on the surrounding anatomy of the PV antrum. The advantage of this approach is that all anatomical variations can be safely treated by moving the ablation plane axially to avoid ablating collateral structures and or by tilting the ablation plane by bending the distal portion of ablation catheter 105.

Numerous other variations and combinations of the features discussed above can be utilized. For example, the emitter structure or transducer array 512 can be slideably mounted within the catheter so that the catheter stays in place during the treatment. In still other arrangements, several emitters might be mounted on the catheter in a chain like fashion in order to apply energy over the length of the catheter inserted into the left atrium. Again this configuration does not require a movement of the catheter during treatment. In still other embodiments, focusing devices, such as lenses and diffractive elements can be employed in case of ultrasonic energy.

The state of the lesion annulus within the PV antrum can be monitored by ultrasound imaging during the treatment. During treatment, the tissue changes its physical properties, and thus its ultrasound reflectivity when necrosed. These changes in tissue ultrasound reflectivity can be observed using ultrasonic imaging to monitor the formation of the desired lesion in the annulus within the PV antrum. Other imaging modalities which can detect heating can alternatively or additionally be used to monitor the treatment. For example, magnetic resonance imaging can detect changes in temperature. In the case of reliance on non-ultrasound imaging modalities, it is optional to include the imaging transducer section 504 as part of the ultrasound transducer array 412.

1. Apparatus for inactivating bronchial nerves in a mammalian subject, comprising:
an electromechanical transducer adapted for insertion into the bronchial tree of the mammalian subject and for emitting unfocused ultrasonic mechanical vibratory energy; and
an energization circuit electrically connected to the transducer, the energization circuit being adapted to control the electromechanical transducer to transmit unfocused ultrasonic mechanical vibratory energy into a target region of at least approximately 1 cubic centimeter, encompassing a section of a bronchial branch so that the unfocused ultrasonic mechanical vibratory energy as applied is effective to inactivate conduction of bronchial nerves and ablate smooth muscle throughout the target region, and insufficiently great to cause tissue necrosis.

2. The apparatus of claim 1, wherein the energization circuit is adapted to control the electromechanical transducer to transmit unfocused ultrasonic mechanical vibratory energy at a power of approximately ten watts for approximately ten seconds to provide an absorbed dose of approximately 100 joules in the target region.

3. The apparatus of claim 1, wherein the energization circuit is adapted to control the transducer so as to maintain the temperature of the bronchial wall below 65° C. while achieving a temperature above 42° C. throughout the target region surrounding the bronchial section treated.

4. (canceled)

5. The apparatus of claim 1, wherein the electromechanical transducer is a circular phased array and the energization circuit is adapted to generate ultrasound imaging and treatment signals or waveforms that are interleaved or intercalated.

6. (canceled)

7. (canceled)

8. The apparatus of claim 1, further comprising a catheter with a distal end and a proximal end, the transducer being mounted to the catheter adjacent the distal end inside a compliant bladder, filled with circulating fluid, in order to cool, center and align the transducer with an axis of the bronchial section treated.

9. (canceled)

10. (canceled)

11. (canceled)

12. (canceled)

13. The apparatus of claim 1, wherein the electromechanical transducer is further adapted to receive ultrasound signals representing the bronchial geometry and the energization circuit is further adapted to:
control the electromechanical transducer to transmit measurement ultrasonic mechanical vibratory energy at a level below the therapeutic level, receive echo signals from the transducer representing reflected measurement ultrasonic energy;
analyze the received echo signals; and
determine a size of the bronchial section to be treated based on the received echo signal.

14. (canceled)

15. A method for inactivating bronchial nerves in a mammalian subject, comprising the steps of:
inserting an electromechanical transducer into a bronchial section of the mammalian subject; and
energizing the transducer to transmit a therapeutically effective dose of unfocused ultrasonic mechanical vibratory energy into an target region of at least approximately 1 cubic centimeter, encompassing the bronchial section so that the unfocused ultrasonic mechanical vibratory energy inactivates conduction of all the bronchial nerves in the target region.

16. The method of claim 15, wherein the ultrasonic mechanical vibratory energy is transmitted at a power of approximately ten watts for approximately ten seconds to provide an absorbed dose of approximately 100 joules throughout the target region.

17. The method of claim 15, wherein the step of transmitting ultrasonic mechanical vibratory energy is performed so as to maintain the temperature of the bronchial wall below 65° C. while heating the bronchial nerves in the target region to above 42° C.

18. (canceled)

19. (canceled)

20. (canceled)

21. (canceled)

22. The method of claim 15, wherein the electromechanical transducer is a phased array, further comprising the step of performing imaging and treatment substantially contemporaneously.

23. (canceled)

24. (canceled)

25. (canceled)

26. (canceled)

27. (canceled)

28. The method of claim 15, wherein the step of inserting the electromechanical transducer is performed over a guide wire which has been placed through the working channel of a bronchoscope.

29. The method of claim 15, further comprising the steps of:
applying non-therapeutic ultrasonic mechanical vibratory energy at a power level less than a power level of the therapeutically effective ultrasonic energy;
receiving reflected non-therapeutic ultrasonic energy and generating encoded echo signals in response to the reflected energy; and
determining a size of the bronchial section to be treated based on the encoded echo signals before the step of energizing the transducer to apply the therapeutically effective ultrasonic energy.

30. (canceled)

31. (canceled)

32. (canceled)

33. A probe for use in bronchial nerve and smooth muscle inactivation, the probe comprising:
an electromechanical transducer adapted for emitting unfocused ultrasonic mechanical vibratory energy;
a catheter with a distal end and a proximal end, the transducer being mounted to the catheter adjacent the distal end; and
an expandable bladder attached at least indirectly to the catheter, the transducer being disposed inside the bladder, the bladder being inflatable by introduction therein of a fluid so that the bladder contacts a wall of a bronchial section to position the distal end of the catheter and the transducer within the bronchial section, which is to be treated.

34. (canceled)

35. The probe of claim 33, wherein the transducer has an axis, the catheter is constructed and arranged to hold the axis of the transducer generally parallel to the axis of the bronchial section to be treated, and the transducer is adapted to transmit the ultrasonic mechanical vibratory energy in a 360° cylindrical pattern surrounding the axis of the transducer.

36. The probe of claim 33, wherein the catheter includes a centering element configured to hold the transducer substantially centered in the bronchial section to be treated.

37. (canceled)

38. (canceled)

39. A medical apparatus comprising an elongate flexible tubular member provided along a distal end portion with an array of electromechanical transducers configured for dual mode imaging and soft-focus ultrasound denervation, said distal end portion including a sandwiched multilayer structure including said array as a first layer, and at least one impedance matching layer disposed over or atop said first layer.

40. The apparatus as set forth in claim 39, further comprising energizing circuitry operatively connected to said array for selectively activating said transducers as a phased array to focus ultrasound energy and obtain imaging data, said circuitry including multiplexer circuits disposed in a staggered fashion at or proximate said distal end portion.

41. An apparatus as set forth in 39 wherein said sandwiched multilayer structure includes, along part of an axial length thereof, reflective backing for therapeutic mode optimization and further includes, along another part of said axial length, absorptive backing for imaging mode optimization.

42. An apparatus as set forth in 39 wherein said array is in the form of a flat rotatable disc, divided into imaging and therapy portions respectively having absorptive and reflective backing.

43. A minimally invasive surgical method comprising:
(a) providing a catheter assembly having a distal end portion carrying a balloon structure and an array of electromechanical transducer elements therein;
(b) inserting a segment of said catheter assembly into a patient so that said distal end portion is disposed inside a preselected tubular organ of the patient;
(c) inflating said balloon structure with a liquid;
(d) obtaining an image of internal organic structures of the patient in a region including said preselected tubular organ;
(e) positioning said distal end portion and said balloon structure in said preselected tubular organ; and
(f) activating said array to transmit unfocused ultrasonic mechanical vibratory energy into a target region of at least approximately 1 cubic centimeter, encompassing a section of the preselected tubular organ so that the unfocused ultrasonic mechanical vibratory energy as applied is effective to inactivate conduction of nerves and ablate smooth muscle throughout the target region, and insufficiently great to cause tissue necrosis.

44. (canceled)

45. (canceled)

46. A method as set forth in claim 43 wherein said image is an ultrasound image and said array is selectively configured for dual mode operation including imaging and unfocused ultrasonic emission, the obtaining of said image including poling transducer elements of said array to detect reflected ultrasonic pressure waves.

47. (canceled)
